(19) United States
(12) Patent Application Publication
(10) Pub. No.: US 2017/0242455 A1
(43) Pub. Date: Aug. 24, 2017

(54) Sterile Screen Protector

(71) Applicant: Shavena Dickens, Nashville, NC (US)

(72) Inventor: Shavena Dickens, Nashville, NC (US)

(21) Appl. No.: 15/052,864

(22) Filed: Feb. 24, 2016

(51) Int. Cl. G06F 1/16; A61B 46/10

(52) U.S. Cl. CPC G06F 1/1607; A61B 46/10

(57) ABSTRACT

A sterile screen protector includes a frame that may be removably coupled to a touch screen in a surgical environment. A sheet is coupled to the frame. The sheet covers the touch screen when the frame is coupled to the touch screen. Thus, a sterilized user may manipulate the touch screen without comprising sterility of the sterilized user. An adhesive layer is coupled to the frame. The adhesive layer adhesively engages the touch screen thereby facilitating the frame to be removably retained on the touch screen.

### BACKGROUND OF THE DISCLOSURE

### Field of the Disclosure

The disclosure relates to screen protector devices and more particularly pertains to a new screen protector device for facilitating a sterilized user to manipulated a touch screen in a surgical environment.

### SUMMARY OF THE DISCLOSURE

An embodiment of the disclosure meets the needs presented above by generally comprising a frame that may be removably coupled to a touch screen in a surgical environment. A sheet is coupled to the frame. The sheet covers the touch screen when the frame is coupled to the touch screen. Thus, a sterilized user may manipulate the touch screen without comprising sterility of the sterilized user. An adhesive layer is coupled to the frame. The adhesive layer adhesively engages the touch screen thereby facilitating the frame to be removably retained on the touch screen.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a front perspective view of a sterile screen protector according to an embodiment of the disclosure.

FIG. 2 is a back view of an embodiment of the disclosure.

FIG. 3 is a cross sectional view taken along line 3-3 of FIG. 2 of an embodiment of the disclosure.

FIG. 4 is a front view of an embodiment of the disclosure.

FIG. 5 is a perspective in-use view of an embodiment of the disclosure.

### DESCRIPTION OF THE PREFERRED EMBODIMENT

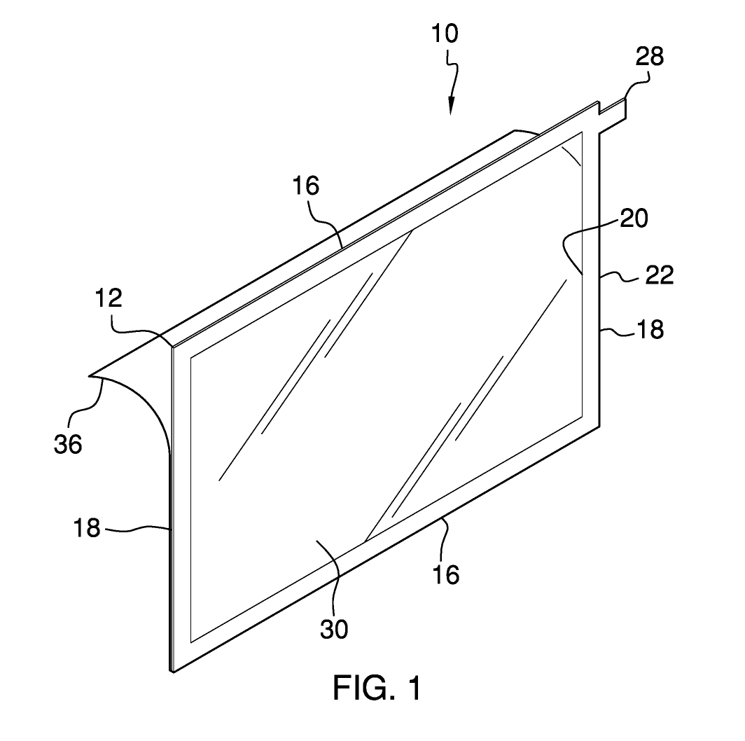

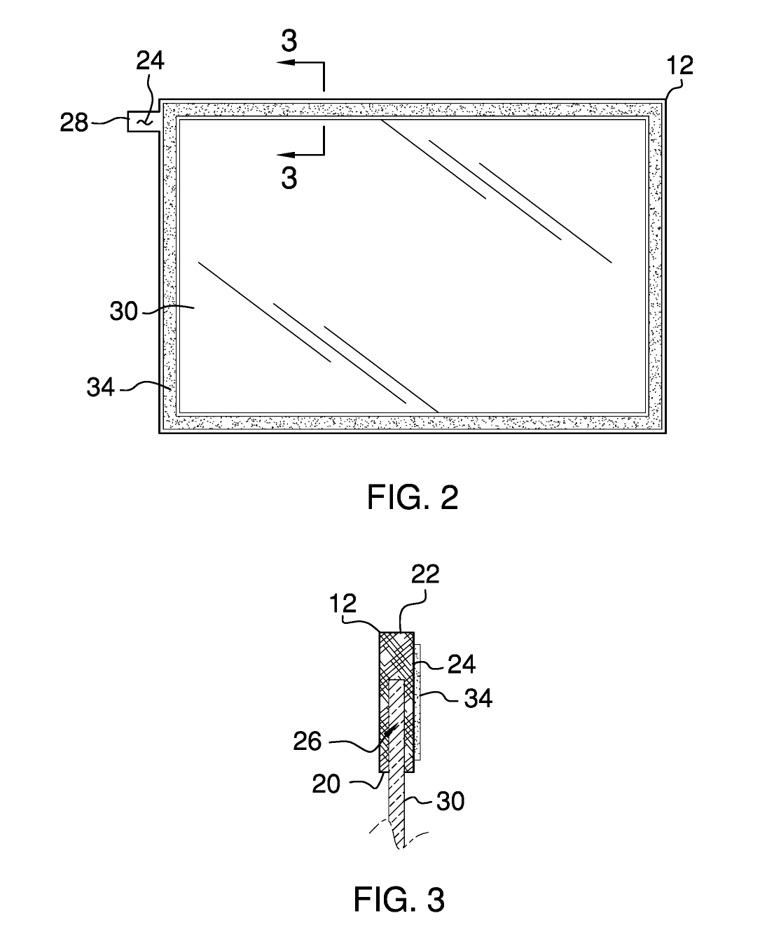

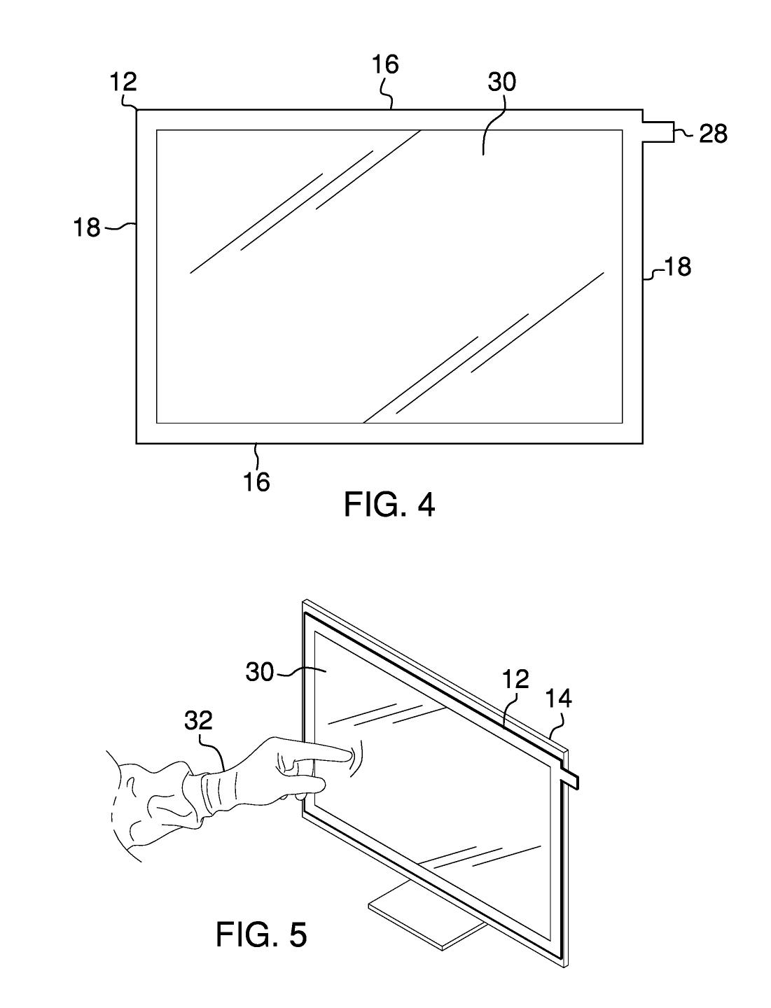

With reference now to the drawings, and in particular to FIGS. 1 through 5 thereof, a new screen protector device embodying the principles and concepts of an embodiment of the disclosure and generally designated by the reference numeral 10 will be described.

As best illustrated in FIGS. 1 through 5, the sterile screen protector 10 generally comprises a frame 12 that may be removably coupled to a touch screen 14 in a surgical environment. The frame 12 has a pair of lateral arms 16 extending between a pair of longitudinal arms 18. The lateral arms 16 are spaced apart from each other such that the frame 12 has an open rectangular shape. The touch screen 14 may comprise medical equipment or the like.

The frame 12 has an inwardly facing edge 20, an outwardly facing edge 22 and a rear surface 24. The inwardly facing edge 20 has a slot 26 extending toward the outwardly facing edge 22. The slot 20 is coextensive with the frame 12. The outwardly facing edge 22 has a tab 28 extending outwardly therefrom. Thus, the tab 28 may be gripped thereby facilitating the frame 12 to be removed from the touch screen 14. The tab 28 may be positioned on one of the longitudinal arms 18.

The frame 12 is colored in a bright color with respect to the touch screen 14. The frame 12 has visible contrast with respect to the touch screen 14. Thus, an observer may visually confirm that the frame 12 is on the touch screen 14. The bright color may be yellow, orange or red.

A sheet 30 is coupled to the frame 12. The sheet 30 covers the touch screen 14 when the frame 12 is coupled to the touch screen 14. Thus, a sterilized user 32 may manipulate the touch screen 14 without comprising sterility of the sterilized user 32. The sheet 30 is positioned within the slot 20 and the sheet 30 is coextensive with the slot 20. The sterilized user 32 may be a doctor, a nurse, a surgical technician or other sterilized user in the surgical environment.

The sheet 30 is comprised of an electrically conductive material. Thus, the sheet 30 facilitates the touch screen 14 to respond to touch when the sheet 30 covers the touch screen 14. The sheet 30 is comprised of a translucent material. Thus, the sheet 30 facilitates the touch screen 14 to be visible when the sheet 30 covers the touch screen 14.

An adhesive layer 34 is coupled to the frame 12. The adhesive layer 34 adhesively engages the touch screen 14 when the frame 12 is positioned on the touch screen 14. Thus, the frame 12 is removably retained on the touch screen 14. The adhesive layer 34 is positioned on the rear surface 24 and the adhesive layer 34 is coextensive with the frame 12.

A protective layer 36 is removably coupled to the adhesive layer 34. The protective layer 36 protects the adhesive layer 34 prior to the frame 12 being removably coupled to the touch screen 14. The sterile cover assembly 10 is sterilized during the manufacturing process. The sterile cover assembly 10 may be packed in sterile packaging in the convention of surgical instruments. The sterile cover assembly 10 may be removed from the sterile packing in the surgical environment.

In use, the protective layer 36 is removed from the adhesive layer 34 prior to commencing a surgical procedure. The frame 12 is positioned on the touch screen 14 such that said adhesive layer 34 engages the touch screen 14. The sheet 30 is touched to manipulate the touch screen 14. The sheet 30 inhibits the sterilized user 32 from contacting the potentially contaminated touch screen 14. Thus, the sterile integrity of the sterilized user 32 is maintained in the sterile environment. The frame 12 is removed from the touch screen 14 and the frame 12 is disposed of after the surgical procedure is complete.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sterile screen protector assembly configured to cover a touch screen in a surgical environment, said assembly comprising:
a frame being configured to be removably coupled to a touch screen in a surgical environment;
a sheet being coupled to said frame wherein said sheet is configured to cover the touch screen when said frame is coupled to the touch screen thereby facilitating a sterilized user to manipulate the touch screen without comprising sterility of the sterilized user; and
an adhesive layer being coupled to said frame wherein said adhesive layer is configured to adhesively engage the touch screen thereby facilitating said frame to be removably retained on the touch screen.

2. The assembly according to claim 1, wherein said frame has a pair of lateral arms extending between a pair of longitudinal arms, said lateral arms being spaced apart from each other such that said frame has an open rectangular shape, said frame having an inwardly facing edge, an outwardly facing edge and a rear surface, said inwardly facing edge having a slot extending toward said outwardly facing edge, said slot being coextensive with said frame.

3. The assembly according to claim 2, wherein said outwardly facing edge has a tab extending outwardly therefrom wherein said tab is configured to be gripped thereby facilitating said frame to be removed from the touch screen, said frame being colored wherein said frame is configured to have visible contrast with respect to the touch screen thereby facilitating an observer to confirm that said frame is on the touch screen.

4. The assembly according to claim 1, wherein:
said frame has a slot; and
said sheet is positioned within said slot, said sheet being comprised of an electrically conductive material wherein said sheet is configured to facilitate the touch screen to respond to touch when said sheet covers the touch screen, said sheet being comprised of a translucent material wherein said sheet is configured to facilitate the touch screen to be visible when said sheet covers the touch screen.

5. The assembly according to claim 2, further comprising:
said adhesive layer being positioned on said rear surface, said adhesive layer being coextensive with said frame; and
a protective layer being removably coupled to said adhesive layer, said protective layer protecting said adhesive layer prior to said frame being removably coupled to the touch screen.

6. A sterile screen protector assembly configured to cover a touch screen in a surgical environment, said assembly comprising:
a frame being configured to be removably coupled to a touch screen in a surgical environment, said frame having a pair of lateral arms extending between a pair of longitudinal arms, said lateral arms being spaced apart from each other such that said frame has an open rectangular shape, said frame having an inwardly facing edge, an outwardly facing edge and a rear surface, said inwardly facing edge having a slot extending toward said outwardly facing edge, said slot being coextensive with said frame, said outwardly facing edge having a tab extending outwardly therefrom wherein said tab is configured to be gripped thereby facilitating said frame to be removed from the touch screen, said frame being colored wherein said frame is configured to have visible contrast with respect to the touch screen thereby facilitating an observer to confirm that said frame is on the touch screen;
a sheet being coupled to said frame wherein said sheet is configured to cover the touch screen when said frame is coupled to the touch screen thereby facilitating a sterilized user to manipulate the touch screen without comprising sterility of the sterilized user, said sheet being positioned within said slot, said sheet being comprised of an electrically conductive material wherein said sheet is configured to facilitate the touch screen to respond to touch when said sheet covers the touch screen, said sheet being comprised of a translucent material wherein said sheet is configured to facilitate the touch screen to be visible when said sheet covers the touch screen;
an adhesive layer being coupled to said frame wherein said adhesive layer is configured to adhesively engage the touch screen thereby facilitating said frame to be removably retained on the touch screen, said adhesive layer being positioned on said rear surface, said adhesive layer being coextensive with said frame; and
a protective layer being removably coupled to said adhesive layer, said protective layer protecting said adhesive layer prior to said frame being removably coupled to the touch screen.
